(19) United States
(12) Patent Application Publication
(10) Pub. No.: US 2024/0285764 A1
(43) Pub. Date: Aug. 29, 2024

(54) SONOSENSITIVE COMPOSITION

(71) Applicants: Sorbonne Université, PARIS (FR); Centre National de la Recherche Scientifique, PARIS (FR); Institut National de la Santé et de la Recherche Médicale, PARIS (FR)

(72) Inventors: Nicolas TAULIER, PARIS (FR); Fatima EL HAJJ, CACHAN (FR)

(21) Appl. No.: 18/573,401

(22) PCT Filed: Jun. 21, 2021
(86) PCT No.: PCT/EP2021/066751

§ 371 Date: Dec. 21, 2023

(51) Int. Cl. A61K 41/00; A61K 47/36

(52) U.S. Cl. CPC A61K 41/0047; A61K 47/36

(57) ABSTRACT

The present invention relates to a composition comprising a matrix and a carrier encapsulating a therapeutic compound, wherein the matrix is configured to release the therapeutic compound under an ultrasound stimulus. A wound dressing includes the composition is also disclosed. The therapeutic compound can be released by being exposed to an ultrasound stimulus.

### FIELD OF INVENTION

The present application is in the field of sonosensitive composition, to be used for example as a wound dressing.

The application pertains generally to devices for the protection and treatment of wounds, as well as methods for accelerating the healing of a wound.

### BACKGROUND OF INVENTION

Hydrogels had been used in many biomedical applications such as dressing for wound healing, system for delivery of bioactive agents like small chemical drugs, growth factors, and cell transplantation. Incorporating drugs in hydrogel systems overcome the disadvantages of injecting the patient with high dosage or repeated administration, that may result in poor targeting, short circulation time, and in some cases cause severe side effects or toxicity. These systems enhance the efficacy of treatments, as they reduce the toxicity of drugs, and deliver the required dosage by controlling and localizing the therapeutic delivery to cells and tissues.

However, there is limited number of commercially available hydrogels as a drug delivery system in the market. They offer a conventional release manner of the encapsulated drug that rely on the degradation of the polymer and lack in the temporal control delivery.

External stimuli (e.g. acoustic or magnetic field, light, or radio waves) could be applied on hydrogel in order to control the spatio-temporal delivery of the encapsulated drug by turning it to an on-demand release system.

Several studies succeed to combine ultrasound with a drug delivery system based on hydrogel at different parameters. However, these systems work either by raising the temperature of the hydrogel or by cavitation. The former is responsible for the denaturation of therapeutic compounds bound to the hydrogel as well as unwanted side effects on the treated site, while the latter is known to cause damages to both the hydrogel and the patient, such as cell sonoporation, creation of free radicals, protein denaturation, hemorrhages and burns, rendering it unsafe for the patient and requiring the frequent replacement of the delivery system.

Alternate approaches embed drug carriers into the hydrogel. These carriers can be liposomes or polymer particles. Ultrasound is then used to trigger the drug release from these carriers, using the same mechanisms as previously (i.e. heating for thermosensitive carriers or cavitation). To reduce the unwanted adverse effects of cavitation while keeping its ability to rupture either the hydrogel or the drug carrier, it is possible to localize the cavitation event by encapsulating nano/microbubbles either inside the hydrogel or inside the drug carrier or alternately by encapsulating perfluorocarbon nanodroplets that could be vaporized under the action of ultrasound. The drawbacks of this approach are the complexity in the system manufacturing as well as the decreased volume devoted to the drug encapsulation. For these reasons, many investigations have focused on using ultrasound to induce mild hyperthermia in combination with the use of thermosensitive hydrogel or/and hydrogel containing thermosensitive carriers. In this approach, small to moderate acoustic pressures (of hundreds of kPa to several MPa) are used which avoid the occurrence of cavitation.

There is therefore a need for a drug delivery system which is structurally durable and capable of keeping the therapeutic compound unaltered while being totally safe for the patient.

The inventors of the instant application have found a drug delivery system comprising a matrix and a carrier encapsulating a therapeutic compound, capable of both passively release the therapeutic compound and release it under an ultrasound stimulus without having to rely on temperature rise or cavitation.

Surprisingly, they found that the matrix of the invention is reversibly destabilized by the ultrasound stimulus, allowing the release of its cargo without damage to its integrity. The particular characteristic of the ultrasound stimulus allows the release of the cargo without putting the patient at risk of harm, or destroying the matrix.

### BRIEF DESCRIPTION OF THE INVENTION

The present invention relates to a composition comprising a matrix and a carrier encapsulating a therapeutic compound, wherein the matrix is configured to release the therapeutic compound under an ultrasound stimulus.

In one embodiment, the matrix forms a net having a pore size comprised between about 1 nm and about 15 nm, more preferably from about 4 nm to about 12 nm. Such a pore size is measured based on the Density functional theory (DFT) using a surface area analyzer, such as a Gemini VII 2390. Such pore size allows the matrix to retain large therapeutic compound (which size is larger than the pore size) as well as carriers of 150 nm of diameter that encapsulate smaller therapeutic compound (smaller than the pore size) and control the release rate of the large and small therapeutic compound.

In one embodiment, the matrix comprises a hydrogel configured to release the large therapeutic compound under an ultrasound stimulus.

In one embodiment, the hydrogel is a polysaccharide hydrogel selected from the group comprising negatively charged hydrogel, such as alginate, pectin, gellan gum, chondroitin, and positively charged hydrogel, such as chitosan. More preferably the hydrogel is an alginate hydrogel. Alginate hydrogel has a nanostructure porosity of a few nanometers that make it an excellent carrier for the therapeutic compound.

In one embodiment, the alginate hydrogel is selected from the group comprising calcium alginate, magnesium alginate, strontium alginate and barium alginate hydrogel, more preferably calcium alginate hydrogel. Calcium alginate hydrogel is preferred as Ca2+ is the most effective cation for promoting gelation. Furthermore, the surface charge of the Ca2+ cations yield the hydrogel with a network of strong physical crosslinks, enhancing its durability.

In one embodiment, the carriers comprise a liposome configured to release small hydrophilic therapeutic compound under an ultrasound stimulus.

In one embodiment, the liposome comprises unsaturated lipid such as DOPC, DOPE, POPC, DOPS. A liposome comprising unsaturated lipid is better permeabilized under the action of ultrasound than a saturated one.

Preferably, the liposome comprises DOPC.

In one embodiment, the liposome comprises saturated lipid such as DSPC, DPPC, DMPC, DSPE.

In one embodiment, the liposome comprises cholesterol. The addition of cholesterol to a liposome decreases its sensitivity to the ultrasound stimulus.

Preferably, the liposomes comprising cholesterol comprises DOPC and/or DSPC.

In one embodiment, the molar ratio of cholesterol to lipid is inferior to 1. A limited cholesterol to lipid ratio increases the release of the therapeutic compound under the ultrasound stimulus.

In one embodiment, the carrier comprises an oil nanodroplets system configured to release small hydrophobic therapeutic compound under an ultrasound stimulus.

In one embodiment, the oil nanodroplets system comprises an oily core made of an oil compound, stabilized by a shell made of either lipids, polymers, surfactants, sterols or a combinations of these compounds.

The oil compound is selected from the group comprising mono-, di- or glycerol triesters; derived molecules of glycerol, mono-, di- or tri- or tetra-esters of citric acid; derived molecules from citric acid; fatty acids; acid monoesters fat; sterids; sphingolipids; glycerophospholipids; polyketics; saccharolipids; terpenes; lipids derived from prenol; essential oils; grease substitutes; waxes (triglycerides); and combinations of these abovementioned oil compound.

In one embodiment, the therapeutic compound is a neutrally charged compound.

In one embodiment, the therapeutic compound is selected from the group comprising growth factors, anti-cancer agents, anti-bacterial agents, anti-viral agents, anti-fungal agents, painkillers, depigmentation agents, anti-inflammatory agent, keratolytic agents, restructuring agents, anesthetics, hydrating agent.

In one embodiment, the ultrasound stimulus is ranging from about 0.5 MHz to about 15 MHz. The selected range of frequencies limits or avoids completely the side effects of the ultrasound stimulus, whether it is local hyperthermia and/or cavitation.

In one embodiment, the duty cycle of ultrasound stimulus is comprised between about 1 and about 25%, more preferably about 5 to about 20%. Using such a duty cycle limits the increase of temperature, avoiding altogether the denaturation of the therapeutical compound and the occurrence of harm to the patient.

In one embodiment, the peak-to-peak acoustic pressure of the ultrasound stimulus is comprised between about 1 and about 8 MPa, more preferably between about 2 and about 5 MPa. The selected range of acoustic pressure ensures an optimal release while avoiding altogether the ill effects of a too high acoustic pressure.

In one embodiment, the hydrogel has a pH comprised between 4 and 7.5.

The present invention also relates to a method for activating the drug release of a composition according to the invention, wherein the composition is exposed to an ultrasound stimulus. In one embodiment, the ultrasound stimulus is ranging from about 0.5 MHz to about 15 MHz. The selected range of frequencies limits or avoids completely the side effects of the ultrasound stimulus, whether it is local hyperthermia and/or cavitation.

In one embodiment, the peak-to-peak acoustic pressure of the ultrasound stimulus is comprised between about 1 and about 8 MPa, more preferably between about 2 and about 5 MPa. The selected range of acoustic pressure ensures an optimal release while avoiding altogether the ill effects of a too high acoustic pressure.

In one embodiment, the duration of the ultrasound stimulus is comprised between about 20 and about 60 minutes. In a preferred embodiment of the invention, the duration of the ultrasound stimulus is about 40 minutes.

In one embodiment, the duty cycle of ultrasound is comprised between about 1 and about 30%, more preferably about 5 to about 20%. Using such a duty cycle limits the increase of temperature, avoiding altogether the denaturation of the therapeutical compound and the occurrence of harm to the patient.

In one embodiment, the Mechanical Index of the ultrasound stimulus is lower than 1.9. A lower Mechanical Index ensure that no cavitation-related adverse effect occurs during the stimulation through ultrasounds.

In one embodiment, the ultrasound stimulus is an unfocused ultrasound beam. Unfocused ultrasound stimulus is able to activate the drug release without an elevation of temperature, which in turn avoid the denaturation of the therapeutical compound and the occurrence of harm to the patient.

The present invention also relates to the use of a composition or a wound dressing according to the invention to treat chronic wounds.

In one embodiment, the patient suffers from a comorbidity selected from the group comprising type 2 diabetes, venous insufficiency, peripheral arterial disease, cardiopulmonary and oxygen transport conditions, cancer, immune deficiencies, renal disease, infection, sepsis, fatigue, depression, and dementia.

### Definitions

“About” preceding a figure means plus or less 10% of the value of said figure.
An “acute wound” is defined as a recent wound that has yet to progress through the sequential stages of wound healing: hemostasis, inflammation, tissue formation and remodeling. An acute wound is acquired as a result of an incision or trauma and heals in a timely and orderly manner. Surgically created wounds include all incisions, excisions, and wounds that are surgically debrided. Surgical wounds include all skin lesions that occur as a result of trauma (e.g. burns, falls), as a result of an underlying condition (e.g. leg ulcers), or as a combination of both.
A “chronic wound” is a wound in which the normal process of healing has been disrupted at one or more points in the phases of hemostasis, inflammation, proliferation, and remodeling. For example, chronic wounds often remain in the inflammatory stage for too long. Wounds that do not heal within three months are often considered chronic. In this wound type, there is usually an underlying pathology, which produces a delay in the healing process.
A “Matrix” is a network of crosslinked polymer chains forming a solid. The crosslinks which bond the polymers together are either physical (i.e. hydrogen bonds, hydrophobic interactions, and chain entanglements (among others)) and/or chemical crosslinks (i.e. covalent bonds between polymer strands).
“Mechanical Index>is a unitless ultrasound metric defined by the equation:

$$MI = \frac{PNP}{\sqrt{f}}$$

where PNP is the peak negative acoustic pressure and f is the center frequency of the ultrasound wave.
A “Therapeutic compound” is any compound, substance, drug, medicament, or active ingredient having a therapeutic or pharmacological effect, and which is suitable for administration to a mammal, e.g., a human. The terms also encompass pharmaceutically acceptable and pharmacologically active ingredients of those active agents specifically mentioned herein including but not limited to salts, esters, amides, pro-drugs, active metabolites, analogs and the like. In the context of the invention, a therapeutic compound is considered a “large therapeutic compound” if its size is larger than the pore size of the matrix. Conversely, a therapeutic compound is considered a “small therapeutic compound” if its size is smaller than the pore size of the matrix.
“Unfocused” when referring to an ultrasound stimulus, preferably a burst of ultrasound signals, refers to an ultrasound stimulus that is not focalized (wherein a focalized ultrasound stimulus is an ultrasound stimulus with only one focal point during the whole duration of the stimulus, preferably a burst of ultrasound signals). Within the meaning of the invention, an unfocused ultrasound stimulus, preferably an unfocused burst of ultrasound signals, includes, without being limited to, the following configurations: the ultrasound stimulus may be simultaneously focused at multiple locations (i.e. multifocal stimulus), an ultrasound stimulus composed of several ultrasonic beams successively focused at different locations within the whole duration of the ultrasound stimulation, or the ultrasound stimulus may be any kind of complex structured spatial and temporal ultrasound pattern.
“Ultrasound stimulus” refers to a stimulus in the form of an acoustic pressure wave. In the present invention, an ultrasound stimulus is delivered to the hydrogel and generated with a transducer array composed of a set of at least one element.

### FIGURES

FIG. 1 is a schematic of the ultrasonic set-up.

FIG. 2 is a graph representing the absorbance spectrum of a solution containing 0.1% alginate (gray slashed line), BSA (black continuous line), BSA with 0.1% of alginate (black slashed line), and BSA with 10 mM of calcium (black dotted line).

FIG. 3 is a graph representing the percentage of released BSA entrapped into an alginate gel when ultrasound were applied at a frequency of 0, 0.8, 1.1, and 3.3˜MHz. At these frequencies, the acoustic pressure was respectively of 0, 8, 5, and 2˜MPa. The percentage of released BSA was quantified after a 20 min of insonation (circles). Then, the gel was washed and the released BSA was again measured after a second 20 min of insonation (squares). A last, measurements were performed after the same procedure (diamonds). All experiments were performed at temperature of 22° C.

FIG. 4 is a graphic representation of the increment in released BSA due to ultrasound as a function of insonation time when the peak-to-peak acoustic pressure was 2.5 MPa (circles), compared to an insonation time of 20 min at an acoustic pressure of 5 MPa (square). Measurements were performed at 22° C.

FIG. 5 is a graphic representation of the percentage of released fluorescein as a function of temperature when using liposomes made of either DOPC (top figure) with 0 mol % (circles), 22 mol % (squares), 34 mol % (up pointing triangles) of cholesterol or DSPC (bottom figure) with 0 mol % (circles), 20 mol % (squares), 33 mol % (up pointing triangles) or 44 mol % (down pointing triangles) of cholesterol. The lines represent fits by Eq. 4 for DOPC and Eq. 5 for DSPC. The chi-square analysis gave the following p-values for DOPC: p=0.999 (0 mol %), 0.996 (22 mol %), 0.999 (34 mol %) and for DSPC: p=0.554 (0 mol %), 0.950 (20 mol %), 0.959 (33 mol %) and 0.981 (44 mol %). The inset plot displays the Tm values derived from the fit as a function of the cholesterol mole fraction into the liposome membrane.

FIG. 6A is a graphic representation of the percentage of released fluorescein as a function of acoustic peak-to-peak pressure using liposomes made of DOPC at 22° C. (white circles, white squares) and 37° C. (black circles, black squares) with 0 mol % (white circles, black circles), 22 mol % (white squares, black squares), and 34 mol % (white triangles, black triangles) of cholesterol. The lines are linear fit of the data. A chi-square analysis gave the following p-values, at 22° C.: p=0.576 (- - -), 0.963 (- - -), 0.915 (- - -), at 37° C.: p=0.816 (−), 0.925 (−), 0.999 (−).

FIG. 6B is a graphic representation of the enhancement of fluorescein release, per unit of acoustic pressure, due to ultrasound triggering at frequencies of 0.8, 1.1 and 3.3 MHz.

FIG. 7A is a graph representing the percentage of released fluorescein as a function of acoustic peak-to-peak pressure using liposomes made of DSPC at 37° C. with 0 mol % (up triangles), 20 mol % (circles), 33 mol % (down triangles) or 44 mol % (squares) of cholesterol. The lines are linear fit of the data. A chi-square analysis gave the following p-values: p=0.999 (−), 0.875 (−), 0.953 (−), and 0.999 (−).

FIG. 7B is a graph representing the enhancement of fluorescein release, per unit of acoustic pressure, due to ultrasound triggering at frequencies of 0.8, 1.1 and 3.3 MHz.

### DETAILED DESCRIPTION OF THE INVENTION

In one embodiment of the invention, the alginate concentration of the hydrogel is comprised between about 1 and about 10% w/v. Preferably, the alginate concentration of the hydrogel is about 3%.

In one embodiment of the invention, the hydrogel is obtained through a reticulation between alginate and a multivalent ion. The molar ration of multivalent ion to alginate is inferior to 3. Preferably, the composition of the composition according to the invention does not contain any compound liable to cause pain during or after application, such as alcohols.

In another embodiment of the invention, the composition comprises a humectant, preferably selected from the group comprising glycerol, urea, simple sugars, hyaluronic acid and its salt, pidolic acid and its derivative, or a combination thereof.

In another embodiment of the invention, the composition comprises an emollient selected from the group comprising vegetal oil, hydrogenated or not, mineral oil such as paraffin, lanolin and their derivatives, vegetal extracts such as Avena sativa, Aloe vera, Centella asiatica or a combination thereof.

In another embodiment of the invention, the composition comprises a pH adjuster selected from the group comprising citric acid, acetic acid, chlorohydric acid, sodium hydroxide or a combination.

The pH of the hydrogel is preferably comprised between 4 and 7.5, more preferably between 5 and 7, even more preferably between 5.5 and 6.5. This range is especially advantageous since it matches the pH of human skin and averts the disruption of the protective function of skin which would slow healing.

The therapeutic compound according to the invention is selected from the group comprising growth factors, anti-cancer agents, anti-bacterial agents, anti-viral agents, anti-fungal agents, painkillers, depigmentation agents, anti-inflammatory agents, keratolytic agents, restructuring agents, anesthetic agents, hydrating agents.

Growth factors according to the invention can be selected from the group comprising Adrenomedullin, Angiopoietin, Autocrine motility factor, Bone morphogenetic proteins (BMPs), Ciliary neurotrophic factor family, Colony-stimulating factors, Epidermal growth factor (EGF), Ephrins, Erythropoietin (EPO), Fibroblast growth factor, Foetal Bovine Somatotrophin (FBS), GDNF family of ligands, Growth differentiation factor-9 (GDF9), Hepatocyte growth factor (HGF), Hepatoma-derived growth factor (HDGF), Insulin, Insulin-like growth factors, Interleukins, Keratinocyte growth factor (KGF), Migration-stimulating factor (MSF), Macrophage-stimulating protein (MSP), Myostatin (GDF-8), Neuregulins, Neurotrophin, Placental growth factor (PGF), Platelet-derived growth factor (PDGF), Renalase (RNLS), T-cell growth factor (TCGF), Thrombopoietin (TPO), Transforming growth factors (TGF), Tumor necrosis factor-alpha (TNF-α), Vascular endothelial growth factor (VEGF), Wnt Signaling Pathway.

Anti-cancer agent according to the invention can be selected from the group comprising alkylating antineoplastic agent and antimetabolite.

Anti-bacterial agents according to the invention can be selected from the group comprising polymyxin B, penicillin such as amoxicillin, clavulanic acid, tetracycline, minocycline, chlortetracycline, aminoglycosides, amikacin, gentamicin, neomycin, silver and its salt such as silver sulfadiazine, and probiotic.

Anti-viral agents according to the invention can be selected from the group comprising acyclovir, famciclovir and ritonavir.

Anti-fungal agents according to the invention can be selected from the group comprising polyenes, Nystatin, Amphotericin B, Natamycin, imidazole such as Miconazole, Ketoconazole, Clotrimazole, Econazole, Bifonazole, Butoconazole, Fenticonazole, Isoconazole, Oxiconazole, Sertaconazole, Sulconazole, Thiabendazole, Tioconazole, triazoles such as Fluconazole, Itraconazole, Ravuconazole, Posaconazole, Voriconazole, allylamines, Terbinafine, Amorolfine, Naftifin, Butenafine; Flucytosine, Griseofulvin, Caspofungin, and Micafungin.

Painkillers according to the invention can be selected from the group comprising paracetamol, codeine, dextropropoxyphene, tramadol, morphine and its derivatives, and corticoids and its derivatives.

Anti-inflammatory agents according to the invention can be selected from the group comprising glucocorticoids, non-steroidal anti-inflammatory, Aspirin, Ibuprofen, Ketoprofen, Flurbiprofen, Diclofenac, Aceclofenac, Ketorolac, Meloxicam, Piroxicam, Tenoxicam, Naproxen, Indomethacin, Naproxcinod, Nimesulide, Celecoxib, Etoricoxib, Parecoxib, Rofecoxib, Valdecoxib, Phenylbutazone, niflumic acid, mefenamic acid, and beta-18-glycyrrhetinique acid.

Depigmentation agents according to the invention can be selected from the group comprising kojic acid, arbutin, a combination of sodium palmitoylpropyl and of white water lily extract, undecylenoyl phenylalanine, licorice extract obtained by fermentation of Aspergillus and ethoxydiglycol, octadecenedioic acid, alpha-arbutin, SACI-CFPA, Arctophylos Uva Ursi leaves aqueous extract, diacetyl boldine, Japanese tangerine extract, kojic dipalmitate, Vegewhite® of LCW, wheat germ extracts and ethyldiamine triacetate.

Keratolytic agents according to the invention can be selected from the group comprising salicylic acid, zinc salicylate, ascorbic acid, alpha hydroxy acid such as glycolic acid, lactic acid, malic acid, citric acid and tartaric acid, silver maple extract, sour cherry extracts, tamarind extracts, urea, topic retinoid, proteases obtained from fermentation of Bacillus subtilis, Linked-Papain® (SACI-CFPA), and papain.

Restructuring agents according to the invention can be selected from the group comprising silica derivatives, vitamin E, chamomile extract, calcium, Equisetum arvense extract, and silk Lipester. Anesthetic agents according to the invention can be selected from the group comprising benzocaine, lidocaine, dibucaine, pramoxine hydrochloride, bupivacaine, mepivacaine, prilocaine, and etidocaine.

Hydrating agents according to the invention can be selected from the group comprising glycerin and vitamins.

The composition according to the invention may also contain perfume or bitterness agent such as denatonium benzoate.

The composition according to the invention may also contain additives, such as preservatives. In a preferred embodiment, the ultrasound stimulus is ranging from about 0.5 MHz to about 15 MHz, preferably from about 1 to about 5 MHz, more preferably from about 1 to about 3.5 MHz. In an alternative embodiment, the lower range of the ultrasound stimulus is of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14 or 14.5 Mhz.

In an alternative embodiment, the upper range of the ultrasound stimulus is of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15 Mhz.

In one embodiment, the ultrasound stimulus is of about 0.5 MHz, preferably of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15 MHz. In one embodiment, the ultrasound stimulus is of about 1 MHz.

In one embodiment, the ultrasound stimulus has a peak-to-peak acoustic pressure comprised between about 1 and about 8 MPa, preferably between about 2 to about 5 MPa. In one embodiment, the ultrasound stimulus has an acoustic pressure of about 1 MPa, preferably of about 1.5, 2, 2.5, 3, 3.5, 4, 4.5, 5, 5.5, 6, 6.5, 7, 7.5 or 8 MPa. In one embodiment, the ultrasound stimulus is of about 2.5 or 5 MPa.

In one embodiment, the ultrasound stimulus has a duty cycle (DC) comprised between 1 and 25%, more preferably 5 to 20%. In one embodiment, the duty cycle of the ultrasound stimulus is about 1%, preferably about 2, 3, 4, 5, 6, 7, 8, 9, 10, 11, 12, 13, 14, 15, 16, 17, 18, 19 or 20%. In one embodiment, the duty cycle of the ultrasound stimulus is about 5%.

In one embodiment, the ultrasound stimulus is unfocused.

The wound dressing according to the invention is bound to be used on skin tissue, typically on areas greater than 1 cm², or even greater than 100 cm².

Preferably, the wound dressing is to be applied on wounds or scars, whether they are caused by an accident, an illness, a burn or as a consequence of a surgery. The wound to be treated is either an acute wound or a chronic wound.

The composition according to the invention is applied on any ailment affecting skin, such as acne, varicella, zona, rosacea, first degree burn, eczema, hyperpigmentation, polymorphous sunlight eruption, vitiligo, xerosis, porphyria, stretch marks, psoriasis, or insect bites. The composition according to the invention can equally be applied to blisters, chaps, or crevasses.

The liposome is firstly obtained by methods known to the person skilled in the art, such as thin film hydration method, in presence of the therapeutic compound to encapsulate. The liposome solution is then mixed with in a hydrogel precursor solution, for example a sodium alginate solution. The hydrogel is then reticulated through the addition of a multivalent ion solution, before being processed to obtain a composition.

Other advantages and features of the present invention will become readily apparent from the following detailed description of the invention. The following figures and examples are put forth so as to provide those of ordinary skill in the art with a complete disclosure and description of how to make and use the present invention, and are not intended to limit the scope of what the Inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### EXAMPLE

The present invention is further illustrated by the following examples.

A—Ultrasound-triggered release of proteins encapsulated into an alginate gel

### 1 Material and Methods

### 1. 1 Materials

Sodium alginate, gelatin type A, calcium carbonate, glutaraldehyde, D-(+)-Gluconic acid gammalactone, Bovine Serum Albumin were purchased from Sigma-Aldrich. Water was purified using a PURELAB Option-Q unit (from ELGA LabWater). Calcium ionic probe was purchased from VWR.

### 1.2 Experimental Design

Alginate and gelatin hydrogels were prepared and loaded with a model protein BSA. Alginate hydrogel was crosslinked thanks to electrostatic Ca²⁺ bonds, while gelatin was crosslinked with glutaraldehyde. Both hydrogels were exposed to ultrasound in order to study the possibility to enhance the release of the protein at frequencies of 0.8, 1.1, and 3.3 MHz and low acoustic pressure without inducing cavitation neither temperature elevation. Alginate hydrogel was characterized after insonation by measuring the released quantity of Ca²⁺ ions in order to understand the mechanism behind the release behavior.

### 1.3 Preparation of BSA Solution

Initial BSA solution was prepared at 100 mg/mL, then dialyzed for 5 hours using dialysis tubing (Dialysis tubing, MWCO 12400, SIGMA-ALDRICH) in distilled water to eliminate the small molecular weight substances that may interfere with the experimental works.

The volume of BSA solution after dialysis changed, hence, the necessity to quantify the new concentration. The absorbance of a substance reflects the concentration according to Beer-Lambert law (A=epsilon×L×C). The amino acids tryptophan and tyrosine present in BSA absorb light at 280 nm. So we measured the absorbance of dialyzed BSA (diluted 30× to avoid spectrum saturation) in the range of 230-340 nm with UV-Vis-spectrophotometer (JASCO model V-650), then the mass concentration was derived from the equation (1) of Beer-Lambert:

$$C = \frac{A}{ϵ \times L} \times M \times Dilution factor$$

Where A is the absorbance of BSA, epsilon is the molar attenuation coefficient of albumin which is equivalent to 43824 M⁻¹ cm⁻¹, L is the optical path length which was 1 cm, M is the molecular weight of albumin which is equivalent to 66463 g/mol, and the dilution factor D was equal to 30.

### 1.4 Preparation of Alginate Hydrogel

Initial solution of sodium alginate was prepared at concentration of 5% (w/v) in distilled water and was vortexed for few minutes until fully dissolved. Then, 30 mM of CaCO₃ and dialyzed BSA (diluted 30× in the alginate final mixture) was added to alginate solution and adjusted with distilled water to alginate final concentration of 3% (w/v). The obtained solution was vortexed and degassed using an ultrasonic bath to remove the bubbles. After that, a fresh aqueous glucono-d-lactone (GDL) solution of a concentration doubled to the concentration of CaCO₃ (60 mM) was added to the mixture solution to regulate the pH to neutral (pH=7) in the gel and initiate the gelation. The solution was vortexed gently for 20 sec, and a volume of 400 μL was poured into Teflon molds. The gels were stored at room temperature for 4 hours until fully gelation.

### 1.5 Measurement of Pore Size

The pore size of alginate gels was measured using surface area analyzer based on the Density functional theory (DFT) (Gemini VII 2390, American University of Beirut). We dried the gels in a way that carbon dioxide molecules replaced the water molecules within the gel to keep the gel network intact as following: the samples were dehydrated in graded series of ethanol in distilled water (10, 25, 50, 75, 90, and 100%) for 2 minutes each, then dried using carbon dioxide at a critical point dryer.

### 1.6 Preparation of Gelatin Hydrogel

Initial gelatin solution was prepared at 5% (w/v). 3.3% (v/v) of dialyzed BSA of initial concentration of 100 mg/mL and 0.5% (v/v) of glutaraldehyde was added to the gelatin solution and adjusted with distilled water to gelatin final concentration of 4% (w/v). Then, 400 μL of the mixture was poured in Teflon molds and incubated at room temperature. The color of the gels became yellow when the gelatin and glutaraldehyde crosslinked. It was enough to wait 2 hours until fully gelation.

### 1.7 Ultrasonic Setup

The hydrogels were exposed to acoustic field generated by a focused ultrasound system. It consisted of a waveform generator whose function was to emit an electrical signal, that was then amplified by a radio frequency amplifier (whose average power was monitor by a wattmeter) and converter into a mechanical signal by a transducer of 81.79 mm diameter×19.05 mm high. The native harmonic frequency of the transducer was 1.1 MHz and we used it at acoustic frequencies of 0.8, 1.1, and 3.3 MHz. The number of cycles of each generated frequency was adjusted in order to obtain a duty cycle of 5%, such as 200, 275, and 825 cycles for the frequencies of 0.8 MHz, 1.1 MHz, and 3.3 MHz respectively. The acoustic field was then propagated in a degassed-water bath container adjusted to 22° C., and was focalized on the sample at a distance of 51.7 mm from the transducer. At the transducer focus, the acoustic pressure varied from 2 to 5 MPa peak-to-peak at 1.1 MHz, 2 MPa at 3.3 MHz, and 8 MPa at 0.8 MHz. The ultrasonic parameters (i.e. frequency, acoustic pressure, insonation duration) used in this study are summarized in Table 1.

### 1.8 Ultrasound Triggered Release

The molds containing the gels (FIG. 1) were covered with 650 μL of distilled water and subjected to the acoustic field at the parameters presented in (Table 1). The released BSA was quantified after insonation with UV-spectrophotometer (JASCO model V-650) using the equation (1). The gels insonified for 20 min (i.e. 0.8 MHz, 8 MPa; 1.1 MHz, 5 MPa; 3.3 MHz, 2 MPa) were incubated for extra 40 min and the diffusion of BSA was quantified every 20 min (i.e. time point: t=40 min and t=60 min). In the other hand, we insonified the gels at the parameter (1.1 MHZ, 2.5 MPa) but for insonation duration of 20 min, 40 min, and 60 min in order to compare the release rate with insonified gels of duration 20 min at the double acoustic pressure (i.e. 1.1 MHz, 5 MPa). Other gels were insonified for 40 min but at the lowest acoustic pressure we have (i.e. 1.1 MHz, 2 MPa) to verify if these ultrasonic parameters could be enough to trigger any release from the gels. To verify if the detected absorbance at 280 nm was for BSA, the absorbance spectrum of a solution of BSA with each substance in the gel (i.e. alginate and calcium ions) and for alginate alone were measured. The chosen concentration of alginate alone or with BSA for spectrum measurement was low (0.1% w/v) to avoid a viscose solution that may saturate the signal.

TABLE 1
Ultrasonic parameters used in this study

| | | | | | |
|---|---|---|---|---|---|
| f (MHz) | 0.8 | 1.1 | 1.1 | 1.1 | 3.3 |
| Pₚₖₚₖ (MPa) | 8 | 5 | 2.5 | 2 | 2 |
| τ (min) | 20 | 20 | 20 | 40 | 20 |
| | | | 40 | | |

### 1.9 Calcium Quantification

Calcium ionic selective electrode (Thermo Scientific, 9720BNW) was used to quantify the released Ca²⁺ ions from the alginate gels to the surrounded solvent after insonation in order to understand the mechanism behind the release of BSA from alginate gels. The electrode was connected to 5-Star Benchtop meter (ORION 5 STAR pH, ISE, Cond, DO Benchtop) using the mV mode, and was then calibrated using the calcium standard (Thermo Scientific, Cat. No. 922006) at concentrations that bracket the expected concentrations of released Ca²⁺ such as 10⁻⁴, 10⁻³, and 10⁻². Ionic Strength Adjuster ISA (Thermo Scientific, Cat. No. 932011) was added to standard and sample solution at concentration of 2% (v/v) to provide a constant background of calcium strength. If the resulting slope from the calibration curve was between 25 and 30 mV when the temperature of the standards was between 20 and 25° C., then the performance of the electrode was guaranteed and the quantification of calcium was performed. 250 μL of the surrounded solution of alginate gels insonified or incubated was diluted 2× with distilled water, 12.5 μL of ISA solution was added to the solution, and was then vortexed. Calcium electrode was placed in the solution, and the reading that appeared on the meter screen was recorded. The obtained calcium concentration was multiplied by the dilution factor (×2). The electrode was rinsed with distilled water before each measurement.

### 2 Results

The inventors found that ultrasound at low acoustic pressure could trigger the release from alginate gels but was unable to trigger the release from gelatin gels. The mechanism behind the release of BSA from alginate gels was due to the destabilization of calcium ions only during insonation but it reformed quickly as soon as ultrasound stopped, then the behavior of the release of BSA recovered to be more like the not-insonified gels.

### 2.1 Quantification of BSA

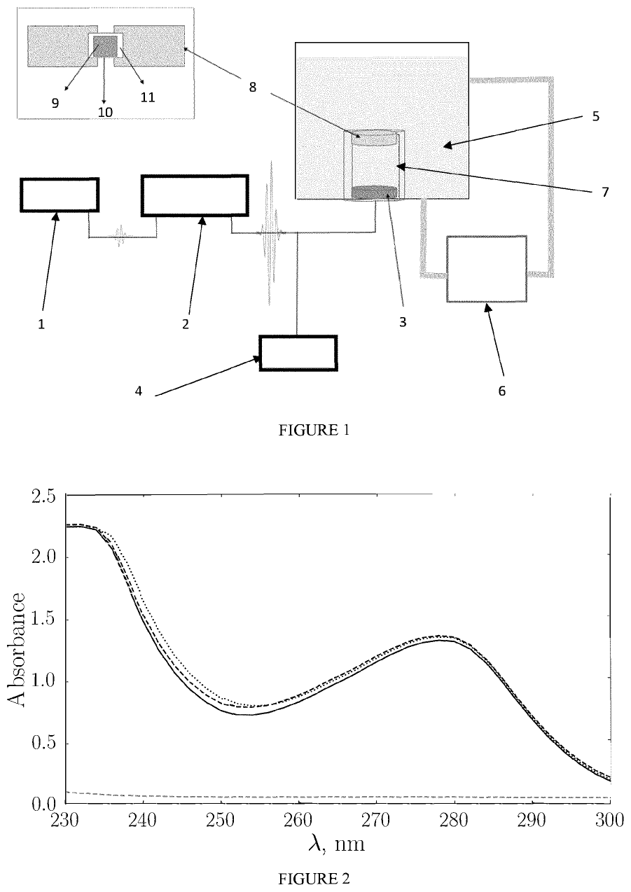

The absorbance of BSA after dialysis was measured in order to determine its final concentration. The absorbance of diluted sample of 30× at 280 nm was equal to 1.29982 which means the final concentration was equal to 59.1 mg/mL (determined using the equation (1)). Therefore, the concentration of BSA presented in the gels was 1.97 mg/mL (59.1 mg/mL×1/30). In order to verify if there was interaction between BSA with alginate or calcium ions, the absorbance spectrum of BSA was measured with either 0.1% alginate or 10 mM of calcium. The absorbance at 280 nm of dialyzed BSA (diluted 30×) with 0.1% w/v of alginate or with 10 mM Calcium was 1.33, and for 0.1% w/v of alginate was 0.04 (FIG. 2). Therefore, the difference in absorbance at 280 nm between BSA alone, and BSA with alginate or with calcium is not significant. Hence, there was no influence of any substance present in alginate gels on BSA quantification.

2.2 Ultrasound Triggered Release from the Gels

The release of BSA from alginate gels immersed in water in the presence or absence of ultrasound was quantified at the time point of 20 min, 40 min, and 60 min at temperature of 22° C. The gels were washed after each incubation to remove all the diffused BSA, and the quantity of BSA left in the gels was considered as total concentration for the following quantification.

In the absence of ultrasound, 11+/−0.7% of the total concentration of BSA presented initially in the gels (1.97 mg/mL) diffused over the first 20 min, then the diffusion decreased in the next 20 min and 40 min (t=40 min and t=60 min) where it became 8+/−1.9% of a total of 1.75 mg/ml and 9.4+/−1.7% of 1.61 mg/mL.

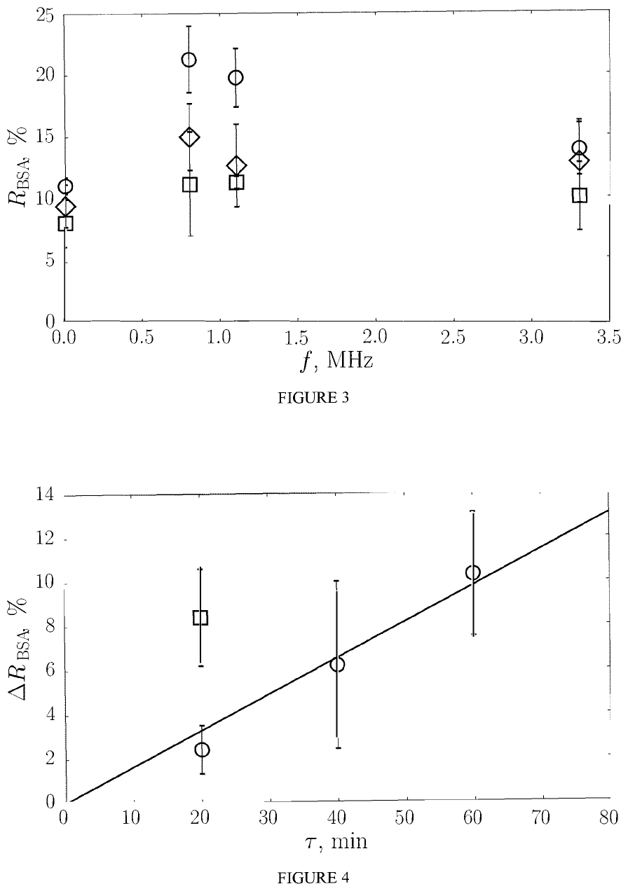

Exposing the gels to ultrasound for 20 min enhanced the release of BSA (FIG. 3. Acoustic field of frequency 0.8 MHz at acoustic pressure of 8 MPa peak-to-peak, increased the release of BSA from gels to around the double (21.3+/−2.7%) of total concentration of BSA in the gels (1.97 mg/mL) comparing to uninsonified gels. While incubation the gels for 20 min post-insonation decreased the release to 11+/−4% of a total concentration of 1.55 mg/mL, then the incubation for a second 20 min post-insonation decreased the release to 15+/−3%) of 1.37 mg/mL. Exposing the gels to larger frequency and lower acoustic pressure (1.1 MHz, 5 MPa) also increased the release of BSA from gels comparing to uninsonified gels and was close to the release from the gels exposed to 0.8 MHz, 8 MPa. However, increasing the frequency to 3.3 MHz decreased the acoustic pressure to 2 MPa. At these parameters (3.3 MHz, 2 MPa), the release of BSA from insonified gels was lower than previous mentioned parameters, 14+/−2% when insonified for 20 min, then it decreased post-insonation to 10+/−3% when incubated for 20 min (t=40 min), and 13+/−3% when incubated for the second 20 min (t=60 min).

In the other hand, insonified gels at acoustic field of frequency 1.1 MHz at low acoustic pressure of 2.5 MPa of an insonation duration of 20 min resulted a release of BSA from gels larger than uninsonified gels of around a rate of 2%. While exposing the gels at the same parameters but for longer insonation duration of 40 min and 60 min increased the release of a rate 6+/−4% and 10+/−3%, which was comparable to the release resulted from insonation at 1.1 MHz, 5 MPa but of 20 min insonation duration. (FIG. 4).

Gelatin gels loaded with BSA were also exposed to ultrasound, but they didn't show any release of the BSA at all the tested ultrasonic parameters.

### 2.3 Calcium Quantification

The detected calcium ions level in the solvent around the gels after insonation at the parameters presented in Table 1 was in the range of 0-0.4 mM (Table 2). The total calcium concentration in alginate gels was 30 mM. The release of calcium ions was less than 1.5% of the total concentration at all the ultrasound exposure parameters. Thus, alginate gels were not destabilized after insonation.

TABLE 2
Quantification of released calcium ions from alginate gels
(1) after 20 min of insonation at several frequencies (0.8
MHz, 1.1 MHz, and 3.3 MHz) and acoustic pressure varying
from 2 MPa to 8 MPa, (2) post-insonation at time point of
40 min and 60 min, and (3) after 40 min of insonation at
acoustic pressure of 2 MPa and 2.5 MPa of frequency 1.1 MHz.

| | | US | Post-US | Post-US | US |
|---|---|---|---|---|---|
| f | Pₚₖₚₖ | 20 min | 40 min | 60 min | 40 min |
| (MHz) | (MPa) | R (mM) | R (mM) | R (mM) | R (mM) |
| 0 | 0 | 0.2 | 0.2 | 0.3 | 0.4 |
| 0.8 | 8 | 0.3 | 0.1 | 0.3 | |
| 1.1 | 5 | 0.2 | 0.1 | 0 | |
| 3.3 | 2 | 0.3 | 0.2 | 0.1 | |
| 1.1 | 2 | | | | 0.4 |
| 1.1 | 2.5 | 0.4 | | | 0.4 |

Placing the alginate gels loaded with BSA in the release medium led to diffusion of BSA through the nanometer pores of the gels (around 11% in the first 20 min) before reaching a stable profile, which is expected in such drug delivery systems. Exposing the gels to acoustic field of several frequencies (0.8, 1.1, and 3.3 MHz) and acoustic pressure varying from 2 to 8 MPa peak-to-peak for several ten minutes (20 min of insonation at the parameters: 0.8 MHz, 8 MPa; 1.1 MHz, 5 MPa, and 40 min of insonation at the parameters 1.1 MHZ, 2.5 MPa) enhanced the diffusion of encapsulated BSA.

The release rate was larger for higher acoustic pressure and lower frequency. Then it decreased as the frequency increased and acoustic pressure decreased. The insonation at the highest frequency 3.3 MHz of acoustic pressure 2 MPa resulted a close release rate from insonified gel at frequency of 1.1 MHz and acoustic pressure of 2.5 MPa (13.5%). Here, the acoustic pressure influenced on triggering the release and not the frequency. However, the release rate from insonified gels at strongest parameters (8 MPa, 0.8 MHz) resulted a close rate to that from insonified gels at the moderate parameters (5 MPa, 1.1 MHz). It is not recommended to use ultrasound at violent parameters to trigger the release from drug delivery system, in addition to that, the strongest parameters in our study didn't contribute to enhancing more the release from the gels comparing to the moderate ones. Another possibility to trigger the release of BSA from alginate gels at low acoustic pressure was by increasing the insonation duration to the double (40 min) and decreasing the acoustic pressure to the half (2.5 MPa peak-to-peak). In this way we obtained a similar release rate to the insonation at 5 MPa for 20 min and was more advantageous since the probability of obtaining a temperature elevation at the treatment site decreased. We recommend avoiding the parameters (8 MPa, 0.8 MHz) for ultrasonic stimulus of drug delivery system and choose instead the moderate ones if the treatment duration has to be short, or the low ones if the treatment duration can be long. As soon as ultrasound stopped, the release rate of BSA from alginate gels decreased and became closer to the diffusion from unexposed gels. We have to note that the drug distribution within the gels changed after insonation and more empty spaces were formed. So BSA was able to travel easier to the surface of the gel then to the surrounded medium. This is the reason why the diffusion in the first 20 min (t=40 min) after insonation was less than the second 20 min (t=60 min). In addition, quantification of calcium ions in the release medium after insonation was negligible (Table 1). For these reasons, we suggest that the mechanism behind the release of BSA from alginate gels was due to destabilization of the calcium bonds only during the application of ultrasound, and they reformed immediately after insonation. Hence, ultrasound turned the alginate gels to an on-demand release system activated only upon insonation.

B—Ultrasound-Triggered Release of Fluorescein from Liposomes

### 3 Materials and Methods

### 3.1 Materials

Solutions of 1,2-dioleoyl-sn-glycero-3-phosphocholine (di18:1 PC or DOPC) and 1,2-distearoylphosphatidyl-ethanolamine (di18:0 PC or DSPC) were purchased from Avanti Polar Lipids as solubilized in chloroform. Cholesterol and phosphate buffer were purchased from Sigma-Aldrich, whereas fluorescein disodium salt (technical grade) was purchased from VWR. The cholesterol assay kit was from Cayman Chemical and the lipid quantification kit was from Cell Biolabs, inc. Water was purified using a PURELAB Option-Q unit (from ELGA LabWater).

### 3.2 Experimental Design

Seven liposome formulations encapsulating sodium fluorescein were prepared either from saturated lipid DSPC mixed with cholesterol at mole fraction of 0, 23, 38, and 48 mol %, or unsaturated lipid DOPC mixed with Cholesterol: 0, 23, and 38 mol %. We determined the liposome hydrodynamic size, the amount of encapsulated sodium fluorescein and the effective [cholesterol:lipid] mole fractions for all the seven formulations. We also studied the passive and ultrasound-triggered of fluorescein at various acoustic pressures and frequencies. For all experiments, the measurements were performed in triplicate.

### 3.3 Liposomes Preparation

Liposomes were prepared by the thin film hydration method. 0, 0.75, 1.5 or 2.25 mg of cholesterol were added to 200 μL of a solution containing 25 mg/mL of either DOPC or DSPC dissolved in chloroform. By doing so, the cholesterol:lipid composition in mol % was estimated to [0:100], [23:77], [38:62], and [48:52]. The four compositions were prepared for DSPC liposomes while only the first three were prepared for DOPC. Next, the lipid mixtures were rotary evaporated under vacuum until dryness. The resulting lipid films were hydrated with 0.5 mL of a solution containing a high concentration of sodium fluorescein (80 mg/mL) dissolved in phosphate buffer solution (PBS) (pH=7.4). The fluorescein concentration was chosen to ensure its quenching. The resulting lipid dispersions were sonicated at 40 W for 5 min with pause cycles of 30 s, and then extruded using polycarbonate membrane of pore size of 200 nm (Avanti, PC membrane 0.2 μm). In order to remove the unencapsulated sodium fluorescein, liposomes suspensions were filtered using microcentrifugal filter tubes (ThermoFisher, Pierce™ Protein Concentrator), containing an ultrafiltration membrane of 100 kDa. These filters retained liposomes while letting the solvent, solubilizing unencapsulated fluorescein, to go through. Before filling the microcentrifugal filter tubes, the samples were diluted 4 times. Then, the tubes were centrifuged at 12,000 g for 1 h 30 min, reducing the volume of liposome solution to 50 μL. The filtered solution was removed then the liposome solution is again diluted and filtered, this process was repeated 4 times to make sure that no free sodium fluorescein remains.

### 3.4 Cholesterol-to-Lipid Ratio

We used two specific kits to separately determine the amount of cholesterol and of lipids for each of our liposome formulation in the absence of fluorescein. The cholesterol assay kit is based on an enzyme-coupled reaction. Cholesterol is first oxidized by cholesterol oxidase to yield hydrogen peroxide and the corresponding ketone product. In the presence of horseradish peroxidase, hydrogen peroxide reacts with 10-acetyl-3,7-dihydroxyphenoxazine in a 1:1 stoichiometry to produce highly fluorescent resorufin. The lipid quantification assay kit measures the neutral lipid content using a lipid binding molecule that fluoresces only when bound to lipids. The enzymatic reaction and the binding of fluorescent molecules were restricted in the case of saturated lipid. Thus, the addition of 10% of Triton X-100 to the suspension followed by a heating at 60° C. (i.e. above the temperature of transition) for 1 h 30 min was necessary to enhance the accessibility of enzymes and fluorescent molecules. Next, liposomes were cooled down at room temperature and diluted 10× or 800× in water for lipid or cholesterol assay, respectively. The resulting fluorescence intensity of both lipid and cholesterol assay were recorded at an emission wavelength of 595 nm using an excitation wavelength of 485 nm. Both cholesterol and lipid amounts were derived from a comparison with a reference curve measured from standard solutions.

### 3.5 Size Measurement

The average hydrodynamic diameter D size along with the Polydispersity Index (PDI) of liposomes in suspension were measured by dynamic light scattering (DLS). Measurements were performed on an ALV/CGS-3 platform-based goniometer system (from ALV GmbH) at 25° C., at scattering angles ranging from 50° to 130° with a step of 20°. At each angle θ, the device provided the decay rate Γθ whose values were plotted as a function of the scattering vector amplitude q(θ)=4πnλ sin(θ/2), where n=1.333 is the refractive index of the solution and λ=633 nm is the laser wavelength. For each sample, the value of D and PDI were obtained from a fit of the curve using the cumulant method.

### 3.6 Fluorescein Release

The passive or ultrasound triggered release of fluorescein was determined by fluorescence spectroscopy using a JASCO spectrofluorometer (model FP 8300). The fluorescence signal coming from the fluorescein encapsulated into the liposomes is quenched due to the high concentration of encapsulated fluorescein. Since all free fluorescein have been previously removed during liposome preparation, any release of fluorescein will be accompanied by an increase in the fluorescence signal. Thus, in these measurements, we first measured the fluorescent spectra FO of the liposome solution before incubation or insonation. The fluorescence intensity in this spectrum is low because of the quenching of encapsulated fluorescein but was not null due to a passive release occurring between the time of liposome preparation and experiments. After an incubation or insonation lasting a time t, the fluorescence spectra F(t) was again measured. At this stage, surfactant Triton X-100 was added to the liposome solution at a concentration of 1%. The surfactant will permeabilize the lipid membrane, leading to the release and dilution of all fluorescein. The fluorescent spectra Flyz of this solution reflects a signal characteristic of the total amount of fluorescein. Consequently, the percentage R of released fluorescein was derived using the equation (2):

$$R = \frac{{F}_{t} - {F}_{0}}{{F}_{lyz} - {F}_{0}} \times 100$$

### 3.7 Fluorescein Load in Liposomes

The quantity of encapsulated fluorescein M_{enc} is the difference between the total amount of added fluorescein during sample preparation Mₜₒₜ and the amount M_{free} of fluorescein that was not encapsulated after liposomes formation. The total amount of added fluorescein was known by weighting and is equal to Mₜₒₜ=40 mg (=80 mg/mL×0.5 mL). After liposome formation, the amount of unencapsulated sodium fluorescein was evaluated from the volume V of the filtered solution going through the first ultrafiltration. The filtered solution was diluted 2000× in PBS (pH 7.4) and its fluorescence intensity was measured from 500 to 550 nm using a JASCO spectrofluorometer (model FP 8300) at an excitation wavelength of 494 nm. The concentration C_{free} of free fluorescein was determined using the fluorescence intensity at 515 nm and a calibration curve previously measured from solutions solubilizing free sodium fluorescein, with a fluorescein concentration ranging from 0.007 to 31.25 μg/mL. The amount of free fluorescein is the amount of fluorescein contained into the filtered solution M_{free}=C_{free}×V.

### 3.8 Ultrasonic Setup

The experiments dealing with insonation were performed using an in-house set-up build specifically for this purpose (FIG. 1). It consisted of a waveform generator 1 (model 33220A from Agilent) that generated an electric signal that was next amplified by radio frequency amplifier 2 (model 150A100C from AR France). Before being converted into an acoustic signal by a focused transducer 3 (model H-101-G from Sonic Concepts), a wattmeter 4 (model NRT from Rhode & Schwarz) continuously monitored the average power. The focused transducer was placed vertically inside a water bath 5 connected to a water degassing and heating unit 6 (model WDS-1005 from Sonic concepts) adjusted at a temperature of 22 or 37° C. A cylinder 7 whose diameter was adjusted to the transducer diameter held a sample holder 8. The cylinder wall contained holes allowing temperature homogenization between the inside and outside volumes. The cell holder was made of a disk where the sample 9 occupied the center. The sample volume (of 1000 mm³) was separated from the bath water by a thin plastic film 10. The sample 9 is surrounded by solvent 11. An ultrasound absorbing material was located one centimeter above the sample holder to avoid ultrasound reflection. We used ultrasonic signals at three frequencies: 0.8, 1.1, or 3.3 MHz. The focus was located partly inside the sample volume and the location was identical between experiments made at the same frequency owed to the set-up design. The focus volume located inside the sample volume differed according to the frequency: its value was 41.75 mm³ at 0.8 MHz, 17.44 mm³ at 1.1 MHz, and 0.87 mm³ at 3.3 MHz. Ultrasound was emitted during 20 min at a duty cycle of 5% and a pulse repetition frequency of 200 Hz. This means that the signal was a repetition pulses made of 200, 275, and 825 sinusoidal cycles when the frequency was respectively 0.8, 1.1, and 3.3 MHz. Finally, the acoustic pressure at the ultrasound focus was measured using a needle hydrophone from Precision Acoustics in the absence of samples and plastic film.

### 3.9 Cavitation Dosimetry

The terephthalate dosimeter is a sensitive technique to detect the occurrence of inertial cavitation when insonation lasts more than a minute, such as in our experiments. Briefly, cavitation creates reactive oxygen species such as hydroxyl radicals OH and hydrogen radicals H. Hydroxyl radicals will bind the non-fluorescent terephthalate (TA) to form the fluorescent hydroxyl-terephthalate (HTA). Consequently, the fluorescence intensity will increase as the number of HTA is formed. While in the absence of cavitation, no radicals will be produced, and the fluorescent intensity will not vary. Thus, the quantity of generated HTA is proportional to the cavitation dose.

In our measurements, 2 mM of TA was prepared in phosphate buffer and maintained at pH 7.3. It is known that the HTA fluorescence signal is linearly proportional to the HTA concentration in the range of 0.2-20 μM. The number of HTA generated under insonation was generally lower than 20 μM in the literature. As a fluorescence reference, we used a 10 HTA solution prepared with the same buffer at a concentration of 1 μM. The fluorescence intensities, F and FO, of the TA solution was measured, at an excitation wavelength of 315 nm and emission wavelength of 422 nm, respectively before and after insonation. The concentration of HTA created due to the occurrence of inertial cavitation is derived from the equation (3):

$${C}_{HTA} = \frac{F - {F}_{0}}{{F}_{ref}} \times {C}_{ref}$$

where C_{HTA} is the concentration of generated HTA and Fref is the fluorescence of the reference HTA solution containing a HTA concentration of C_{ref}=1 μM.

### 4 Results

### 4. 1 Liposomes Properties

We prepared liposomes made of either DSPC or DOPC with various concentrations of cholesterol. All these liposome formulations were prepared such as liposomes encapsulate sodium fluorescein. The fluorescein concentration inside a liposome (around 80 mg/mL at the beginning of the preparation) is large enough to induce its fluorescence quenching. The liposome solution was washed so that negligible free fluorescein was present. Whatever the ratio of cholesterol-to-lipid used into the fluorescein-loaded liposomes, we obtained a similar diameter, that is 175±10 nm and 145±10 nm for liposomes made, respectively, from DSPC and DOPC. In our experiments, we used solutions diluted four times where the concentration of encapsulated sodium fluorescein was about 4.0±0.2 mg/mL for all liposome formulations. For each formulation, the amounts of cholesterol and lipid added to the solution led to the following composition ([mol % cholesterol:mol % lipids]): [0:100], [23:77], [38:62], and [48:52]. Since a loss of materials can be expected after filtration and centrifugation, we derived the composition at the end of the liposome preparation, by separately measuring the quantity of lipids and of cholesterol using specific kits. The error produced by the cholesterol kit was less than 1% and less than 0.1% for the lipid kit. The effective compositions were then [20:80], [33:67], and [44:56] for the last three formulations of DSPC liposomes and [22:78] and [34:66] for the second and third formulation of DOPC liposomes.

### 4.2 Passive Fluorescein Release

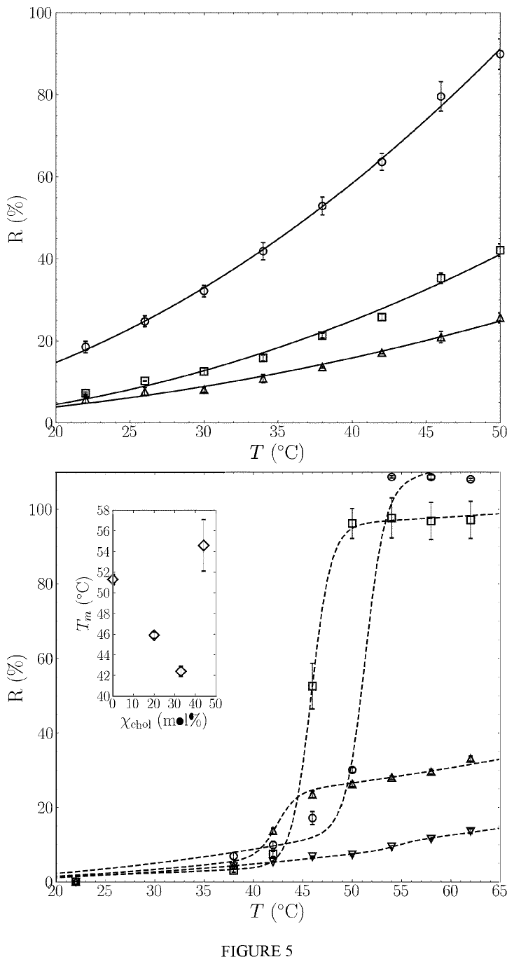

We monitored the passive release for each liposome formulation. To do so, we measured the difference in fluorescence intensity at a time t, then 20 min later. For DOPC formulations, 20 min incubations were performed at 22, 26, 30, 34, 38, 42, 46 and 50° C. The percentage of released fluorescein R was plotted as a function of temperature for DOPC liposomes containing 0, 22 and 34% of cholesterol, represented respectively by blue, red and green circles in the FIG. 5A. For each formulation, the release increased as the temperature rose, but the addition of cholesterol reduced the amount of released fluorescein. The data were well fitted by the following equation (4) (using the least square minimization method):

$$R ( T ) = AT + {BT}^{2}$$

where A and B were constants with no physical significance. For DSPC formulations, higher temperatures were investigated as we knew that the critical temperature of the gel-to-liquid phase transition were around 54.5° C. for liposomes made of only DSPC. Thus, measurements were performed at 22, 38, 42, 46, 50, 54, 58, and 62° C. The data are displayed at FIG. 5B for DSPC liposomes containing 0, 20, 33, and 44% of cholesterol (in blue, red, green, and black squares, respectively). The fluorescein release was small below a critical temperature Tm, then abruptly increased to reach a plateau above Tm, which value decreased as the percentage of cholesterol increased. The R value at 22° C. were below 0.1% for all liposome formulations. The data were fitted using an equation taking into account a transition at a critical temperature Tm (5):

$$R ( T ) = AT + {BT}^{2} + \frac{Δ R}{1 + {e}^{( {T}_{m} - T )}}$$

where ΔR was the release due to the gel-to-liquid transition. From the fit of the experimental data, we derived ΔR and Tm for DSPC liposomes containing respectively 0, 20, 33, and 44 mol % of cholesterol. For ΔR, these values were respectively, (92±10), (91±3), (17±3) and (1.8±1.5)%, while the values of Tm were respectively (51.3±0.5), (45.9±0.3), (42.4±0.5), and (54.6±2.5)° C. (see inset plot in FIG. 5).

### 4.3 Ultrasound-Triggered Release of Fluorescein

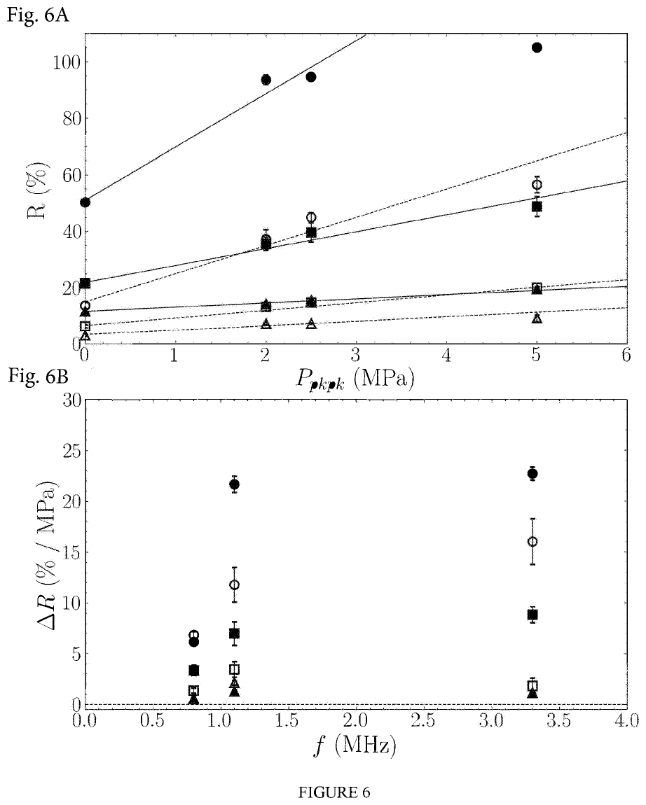

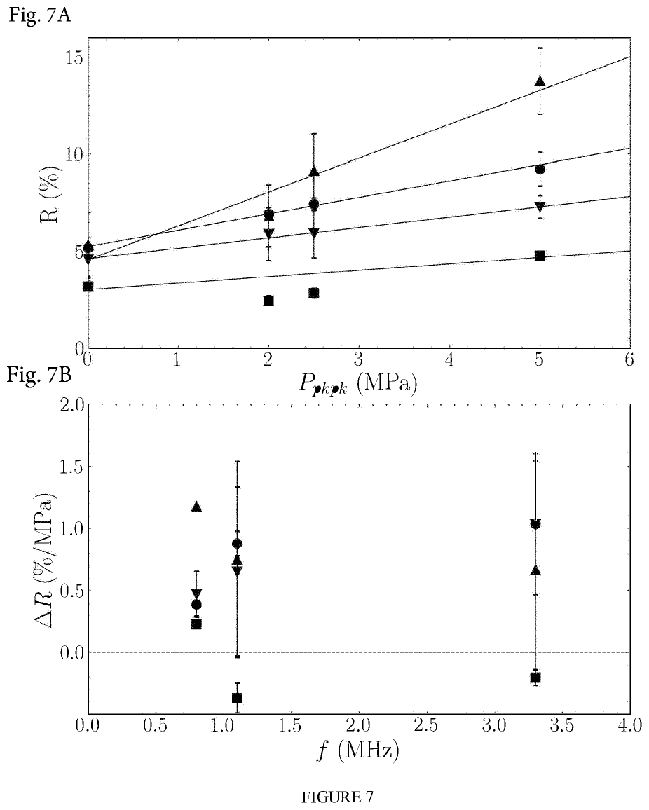

The ultrasound-triggered release experiments were performed at 22 and 37° C. for liposomes containing DOPC and at 37° C. for DSPC liposomes. The temperature 37° C. was chosen because of its physiological relevance. Since the passive release was important (>40%) for DOPC liposomes at 37° C., we also decided to investigate a lower temperature where the passive release was smaller (<20%), that is 22° C., the lower temperature previously explored. Experiments were performed as previously except that an insonation was performed during 20 min instead of a 20 min incubation. For all insonations, the pulse repetition frequency was set to 200 Hz while the duty cycle was kept equal to 5%, i.e. a pulse of 0.25 ms was emitted every 5 ms during the 20 min of insonation. This also means that at each pulse emission, ultrasound was “on” during the first 5% of the time (i.e. during ton=0.25 ms) and “off” during the remaining 95% (i.e. during toff=4.75 ms). The first experiments were performed at an acoustic frequency of 1.1 MHz, which was the fundamental frequency of the transducer used in our setup. Each liposome sample was insonified at different acoustic peak-to-peak pressures: 0, 2, 2.5 and 5 MPa. These pressures were determined in the absence of liposomes using a needle hydrophone. The data for DOPC liposomes are displayed in FIG. 6A. The insonation enhanced the fluorescein release when liposomes were devoid of cholesterol, at 22 and 37° C., or when they contained 22 mol % of cholesterol at 37° C. For the other conditions, i.e at 22 mol % of cholesterol at 22° C. and at 34 mol % of cholesterol at 22 and 37° C., the release enhancement was small. The data for DSPC liposomes are displayed in FIG. 7A. For all conditions except at xchol=20%, a linear increase in fluorescein release was observed as a function of pressure. Next, we investigated the ultrasound-triggered release of encapsulated fluorescein at three frequencies (0.8, 1.1, and 3.3 MHz) as shown in FIGS. 6B and 7B for DOPC and DSPC liposomes respectively. In these figures, we considered ΔR, the pressure-normalized difference in the fluorescein release before and after insonation, since the peak-to-peak pressure was 2 MPa at 1.1 and 3.3 MHz, but 8 MPa at 0.8 MHz. However, we did not normalize these values by the insonified volume despite the fact that this volume decreases as the frequency increases. Indeed, we considered that a 20 min insonation allowed the insonation of all liposomes in the sample volume thanks to diffusion and convection. This assumption was confirmed by the fact we could measure values of 100% for fluorescein release. To make sure that no cavitation occurs, particularly at the lower frequency, we assessed its occurrence using a teraphtalate dosimetry (data not shown). No increase in HTA production was observed (<0.008 UM) at the three frequencies, which confirmed that inertial cavitation did not occurred during our measurements. For liposomes made of DOPC, the percentage of fluorescein release per MPa, ΔR, has decreased as the cholesterol fraction in the liposome increases. Similar ΔR values were obtained at 1.1 and 3.3 MHz while they were smaller at 0.8 MHz. These values confirmed that the insonified volume was not relevant otherwise ΔR would have dramatically decreased. The best values were obtained for liposomes devoid of cholesterol or containing 22 mol % of cholesterol when T=37° C. For DSPC liposomes, ΔR were much smaller as they never exceeded 1.5% during the 20 min of insonation. Surprisingly, negative values were obtained at 20 mol % of cholesterol. During all insonation experiments, only small temperature elevations were measured: that were 0, 0.3, and 1.8° C. at f=1.1 MHz for Ppkpk=2, 2.5 and 5 MPa, respectively, 0.5° C. at f=3.3 MHz and Ppkpk=0.5 MPa, and 2.1° C. at f=0.8 MHz and Ppkpk=8 MPa.

The combination of frequency, pressure and duty cycle values used in our experiments does not lead to inertial cavitation (as measured by the terephtalate dosimeter) and the temperature rise was always contained and never exceeded 2.1° C. It is also informative to calculate the Mechanical Index, defined hereabove. In echography, a MI value higher than 1.9 is prohibited, but a higher value can be used for therapeutic applications such as sono-ablation. In our case, insonation at f=0:8 MHz and Ppkpk=8 MPa gives a MI value of 4.5. At f=1.1 MHz, the values are 0.95, 1.19 and 2.38 for Ppkpk=2, 2.5 and 5 MPa, respectively. Finally, MI=0.55 for f=3:3 MHz and Ppkpk=2 MPa.

Our experimental data show that an insonation better enhances the release of fluorescein encapsulated into a DOPC liposomes than in DSPC liposomes. In the absence of cholesterol, the enhancement difference is more than 20 times between DOPC and DSPC liposomes at 37° C. At this temperature, the membrane of DSPC liposomes remains in the gel or solid ordered phase since the elevation of temperature due to the insonation is not enough to go over Tm, even if this value slightly decreases upon the addition of cholesterol.

The difference in release enhancement becomes small when comparing DSPC formulations and a DOPC liposomes containing 34% of cholesterol. Our data show that ultrasound is more efficient at enhancing the permeability of a membrane in a liquid crystalline or disordered phase than in a gel or liquid ordered phase. Moreover, we do not see difference due to frequency when comparing release measurements performed at 1.1 or 3.3 MHz. Experiments made at 1.1 MHz suggest a linear dependence on acoustic pressure up to 5 MPa. But this may not be true for higher pressures which can explain the smaller ΔR values measured at 0.8 MHz where experiments were performed a Ppkpk=8 MPa. We used a low duty cycle of 5% to reduce heat deposition due to ultrasound. Consequently, during the 20 min insonation ultrasound were effectively “on” only for a cumulated time of 1 min.

Overall, our experimental data confirmed that an insonation lead to an enhancement in the release of fluorescein into liposomes in the absence of cavitation and below Tm in the case of DSPC liposomal formulation. Our data indicate that ultrasounds are more efficient to increase release for liposomes made of unsaturated lipids and this effect is more pronounced in the absence of cholesterol.

The inventors showed that a moderate acoustic pressure induces fluorescein release for all formulations but at different degrees. The highest ultrasound-triggered release is obtained for a liposome made of pure DOPC membrane, but passive release is also important. The lowest ultrasound-triggered release is measured for DSPC liposomes which also exhibit a very small passive release. The fluorescein release is not due to cavitation or heating but to an enhanced diffusion of fluorescein out of the liposome, partially due to an increase in area per lipid as suggested by our MD simulations. This enhanced release diffusion mechanism requires insonations of several minutes but does not requires high pressures, so low mechanical index could be used.

1. Composition comprising a matrix and a carrier encapsulating a therapeutic compound, wherein the matrix is configured to release the therapeutic compound under an ultrasound stimulus.

2. Composition according to claim 1, wherein the matrix forms a net having a pore size comprised between about 1 nm to about 15 nm.

3. Composition according to claim 1, wherein the matrix comprises a hydrogel comprising the therapeutic compound, the hydrogel being configured to release the therapeutic compound under an ultrasound stimulus.

4. Composition according to claim 3, wherein the hydrogel is a polysaccharide hydrogel selected from the group comprising alginate, pectin, gellan gum, chondroitin and chitosan, more preferably is an alginate hydrogel.

5. Composition according to claim 4, wherein the alginate hydrogel is selected from the group comprising calcium alginate, magnesium alginate, strontium alginate and barium alginate hydrogel, more preferably calcium alginate hydrogel.

6. Composition according to claim 1, wherein the therapeutic compound is a neutrally charged compound.

7. Composition according to claim 1, wherein the ultrasound stimulus is ranging from about 0.5 MHz to about 15 MHz.

8. Composition according to claim 1, wherein the peak-to-peak acoustic pressure of the ultrasound stimulus is comprised between about 1 and about 8 MPa, more preferably between about 2 and about 5 MPa.

9. Wound dressing comprising a composition according to claim 1.

10. Method for activating the drug release of a composition according to claim 1 wherein the composition or wound dressing is exposed to an ultrasound stimulus.

11. Method according to claim 10, wherein the acoustic frequency of the ultrasound stimulus is ranging from about 0.5 MHz to about 5 MHz.

12. Method according to claim 10, wherein the acoustic pressure of the ultrasound is comprised between about 1 and about 8 MPa, more preferably between about 2 and about 5 MPa.

13. Method according to claim 10, wherein the duty cycle of ultrasound is comprised between about 1 and about 25%, more preferably about 5 to about 20%.

14. Method according to claim 10, wherein the ultrasound beam is an unfocused ultrasound beam.

15. Use of a composition according to claim 1 to treat chronic wounds.
